(19) United States
(12) Patent Application Publication
(10) Pub. No.: US 2005/0283103 A1
(43) Pub. Date: Dec. 22, 2005

(54) Dressing for a four legged animal

(76) Inventor: Jane Patricia Murfett, Childrev Ox (GB)

Correspondence Address: DAVIS & BUJOLD, P.L.L.C., FOURTH FLOOR, 500 N. COMMERCIAL STREET, MANCHESTER, NH, 03101-1151, US

(21) Appl. No.: 10/521,957

(22) PCT Filed: Jul. 22, 2003
(86) PCT No.: PCT/GB03/03274

§ 371 Date: Jan. 21, 2005

(30) Foreign Application Priority Data
Jul. 22, 2002 (GB) 0216930.8

(51) Int. Cl. A61F 5/00

(52) U.S. Cl. 602/23; 602/75

(57) ABSTRACT

A dressing for a four legged animal comprising: a sheet member containing at least two apertures each adapted to receive a leg and thereby establish an axis of alignment for the dressing relative to a longitudinal axis of an animal to which the dressing is applied; characterised in that the sheet member is bounded by: a first transverse (front) side from which the two apertures are spaced by an amount considerably less than the overall length of the sheet member; a second transverse (rear) side spaced on the axis of alignment and remote from the first transverse side; first and second lateral sides extending from the first transverse side along on either side of the axis of alignment, each lateral boundary of the pair extending from the first transverse side to the second transverse side; the first lateral side of the pair being disposed on one side of the axis of alignment and the second lateral side being disposed on the other side of the axis of alignment to first side; and a locating means defined by, or by way of, the sheet member to provide for location of the sheet member in the vicinity of the second transverse side on a body of an animal to which the dressing is attached to limit movement of the dressing in a direction coaxial with or parallel to the axis of alignment; the dressing being adapted for being retained in a working configuration around the body of an animal with a leg of the animal extending through each of the apertures and a standby configuration where the dressing is available for use prior to wrapping around an animal.

### TECHNICAL FIELD

This invention relates to a dressing that is to say a means serving to provide for protection of a part of a body such as one containing a wound. A dressing can also serve to hold a pad or other item in place relative to a part of the body.

### BACKGROUND ART

The present invention is chiefly, but not exclusively, concerned with a dressing for a four-legged animal. A distinction to be drawn between human bodies and those of four legged animals lies that in a human body arms and legs effectively lie in the same plane as the body whereas in a four legged animal the legs tend to project out of the plane of the body. In providing a dressing to the body of a human it is not usually necessary for the location of arm or legs to be involved. In contrast to this with a four legged animal who has sustained a body injury requiring a major dressing then relating the locating and securing of the dressing relative to one or more of the legs becomes a significant consideration.

In the event of injury to a four legged animal, especially ones relating to a domestic or farming environment, it is common for any surface damage to be treated and dressed to protect the surface area. Such a dressing also serves to prevent the animal having access to the area and through biting or licking aggravate the injury and/or remove or at least diminish the effect of any dressing or medication that has been applied. To this end the dressing needs to be shielded from displacement and so needs to be secured in place. This can have two adverse effects. Firstly having treated the wound the person giving the treatment needs to spend some time achieving protection of the dressing in a way that resists subsequent accidental displacement or tampering. Secondly as a result of securing the dressing the animal can be incapacitated to a greater extent than is justified merely by the nature or extent of the injury.

German Gebrauchsmusterschrift DE 200 05 886 discloses a dressing for dogs and cats with any type of body injury having a one-piece body made of stretchable cotton rep and washable up to 90 degrees C. The body is designed to cover the body of the animal, and has sleeves in the resign of the fore legs and back legs. In the region of the back the body has a Velcro fastener. The body can be manufactured with different measurements to suit different sizes of animal.

### DISCLOSURE OF INVENTION

According to the present invention there is provided a dressing for a four legged animal comprising: a sheet member containing at least two apertures each adapted to receive a leg and thereby establish an axis of alignment for the dressing relative to a longitudinal axis of an animal to which the dressing is applied;
characterised in that the sheet member is bounded by:
a first transverse (front) side from which the two apertures are spaced by an amount considerably less than the overall length of the sheet member;
a second transverse (rear) side spaced on the axis of alignment and remote from the first transverse side;
first and second lateral sides extending from the first transverse side along on either side of the axis of alignment, each lateral boundary of the pair extending from the first transverse side to the second transverse side; the first lateral side of the pair being disposed on one side of the axis of alignment and the second lateral side being disposed on the other side of the axis of alignment to first side; and
a locating means defined by, or by way of, the sheet member to provide for location of the sheet member in the vicinity of the second transverse side on a body of an animal to which the dressing is attached to limit movement of the dressing in a direction coaxial with or parallel to the axis of alignment;
the dressing being adapted for being retained in a working configuration around the body of an animal with a leg of the animal extending through each of the apertures and a standby configuration where the dressing is available for use prior to wrapping around an animal.

According to a first preferred version of the present invention the dressing is further characterised by a complementary linkage means provided on the lateral sides to enable the first lateral side to be demountably coupled to the second lateral side in the working configuration of the dressing and the first lateral side to be freed from coupling with the second lateral side in the standby configuration of the dressing.

According to a second preferred version of the present invention or of the first preferred version thereof the dressing is further characterised in that the second lateral side is shaped so that with the dressing in its working configuration the second lateral side serves to avoid covering a predetermined region of a body of an animal on which the dressing is established in a working configuration.

According to a third preferred version of the present invention or of any preceding preferred version thereof the dressing is further characterised in that the sheet in the region of the second lateral side defines a number of possible paths, such as by marks or lines or perforations in the sheet, the paths providing for the sheet to be cut to establish one of these paths to define a fresh second lateral side for the dressing.

According to a fourth preferred version of the present invention or of any preceding preferred version thereof the dressing is further characterised by a locating means comprising or including a cup or recess defined by, or attached to, the sheet and adapted to receive a body region of an animal on which the dressing is being, or is to be, used in its working configuration.

According to a fifth preferred version of the present invention or of any preceding preferred version thereof the dressing is further characterised in that the location means comprises a tie or loop for attaching to a part of a body of an animal to which the dressing is applied such as a tail.

According to a sixth preferred version of the present invention or of any preceding preferred version thereof the dressing is further characterised in that at least one of the apertures includes a sleeve extending from the aperture so that in the working configuration of the dressing a leg of an animal extending through the aperture is surrounded, and is resiliently held by, the sleeve.

According to a seventh preferred version of the present invention or of any preceding preferred version thereof the dressing is further characterised in that the sheet in addition to the two apertures for first two legs of a four-legged animal provided towards the first transverse side of the sheet there are additionally provided a second pair of apertures for the other two legs of the animal to the first two so that, the dressing is located by the four apertures on the four legs on a subject animal on which the dressing is being, or is to be, used in its working configuration. Preferably at least one second aperture of the second pair of apertures includes a sleeve extending from the second aperture so that in the working configuration of the dressing a leg of an animal extending through the second aperture is surrounded over at least a short length by the sleeve.

According to an eighth preferred version of the present invention or of any preceding preferred version thereof the dressing is further characterised in that the complementary linkage means is chosen from a group comprising: at least one pair of tapes or strings that can be knotted together, a contact material such as VELCRO (RTM), an adhesive or plaster.

According to a ninth preferred version of the present invention or of any preceding preferred version thereof the dressing is further characterised in that the dressing includes means for locating or retaining a local dressing, or means for retaining medication, at a predetermined location in a working configuration of the dressing.

According to a tenth preferred version of the present invention or of any preceding preferred versions thereof the dressing is further characterised in that dressing as claimed in any preceding claim characterised in that the dressing is of a material or materials of a type able to withstand at least one autoclaving or other sterilisation process to enable the dressing to be re-used.

According to an eleventh preferred version of the present invention or of any of the first eight preferred versions thereof the dressing is further characterised in that the sheet material is of a type intended for one of disposable usage.

### BRIEF DESCRIPTION OF DRAWINGS

Exemplary embodiments of the invention will now be described with reference to the accompanying drawing of dressings for use with four legged animals of which:

FIG. 1A shows a plan view of a first embodiment in a ready to use configuration and

FIG. 1B shows the dressing of FIG. 1A located on a male dog;

FIG. 2A shows a plan view of a second embodiment in a ready to use configuration and FIG. 2B shows the dressing of FIG. 2A located on a female dog;

FIG. 3 shows a plan view of a third embodiment ready to be adjusted and fitted; and

FIG. 4 shows a plan view of a fourth embodiment in a ready to use configuration

The dressings are particularly, but not exclusively intended for use for post operative dressing.

### MODES FOR CARRYING OUT THE INVENTION

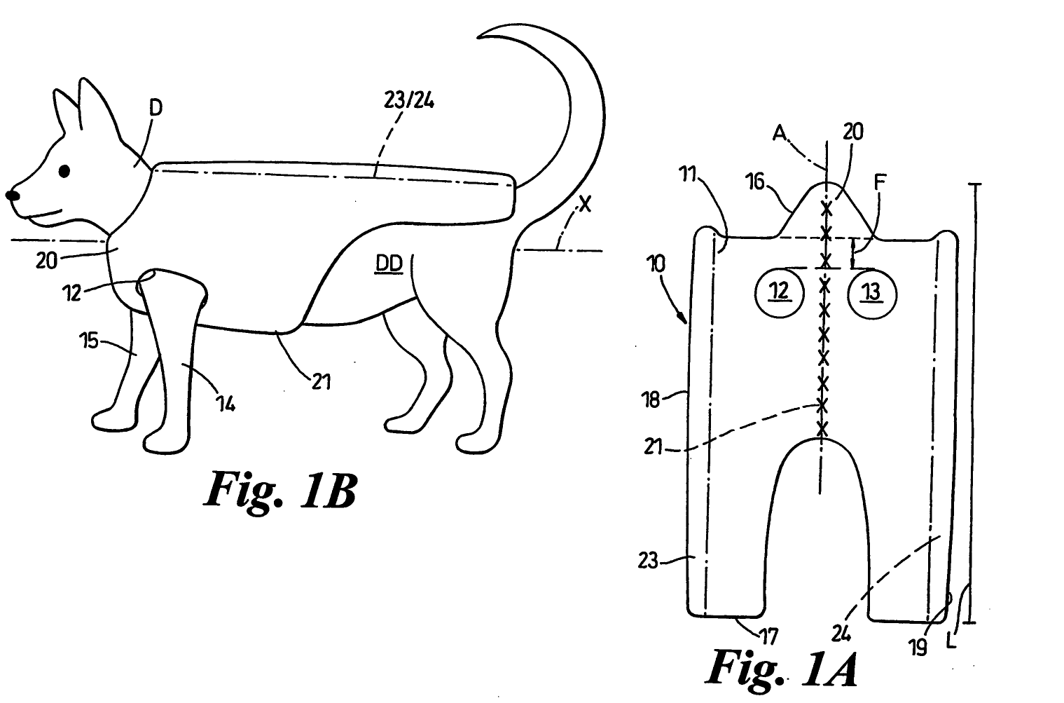

### First Embodiment (FIGS. 1A and B)

These figures variously show a dressing 10 for a male dog D. The dressing 10 comprises: a sheet member 11 containing two apertures 12, 13 each adapted to receive respectively front legs 14, 15 of the dog D. The apertures 12, 13 are disposed symmetrically on either side of axis of alignment A for the dressing 10 relative to longitudinal axis X of the dog D. The sheet member 11 is bounded by:
front transverse side 16 from which the two apertures 12, 13 are spaced by an amount F considerably less than the overall length L of the sheet member 11;
a rear transverse side 17 on the axis of alignment A and at the opposite end of the sheet member 11 to the front transverse side 16;
a pair of lateral sides respectively first lateral side 18 and second lateral side 19 extending from the front transverse side 16 to the rear transverse side 17 and symmetrically disposed along either side of the axis of alignment A.

The sheet member 11 includes a locating means in the form of a cupped section 20 generated in this case by stitching the local part of the sheet member 11. The section 20 serves to provide for location of the sheet member 11 on the body of the dog D to which the dressing 10 is attached to limit movement of the dressing in a direction coaxial with or parallel to the axis of alignment A.

In addition a seam 21 is provided to establish curvature conforming to the underside of the dog D so that in use the dressing 10 is a snug fit.

The dressing 10 is in this case adapted for retention in a working configuration (as shown in FIG. 1B), around the body of dog D with front legs 14, 15 extending, respectively, through apertures 12, 13. The retention is maintained by means of complementary stripes 23, 24 of VELCRO (RTM) on the lateral sides 18, 19.

It will be seen in this first embodiment that the rear transverse side 17 provides for a substantial amount of clearance of rear lower part DD of the dog D typically to enable the dog D to excrete matter without soiling the dressing 10.

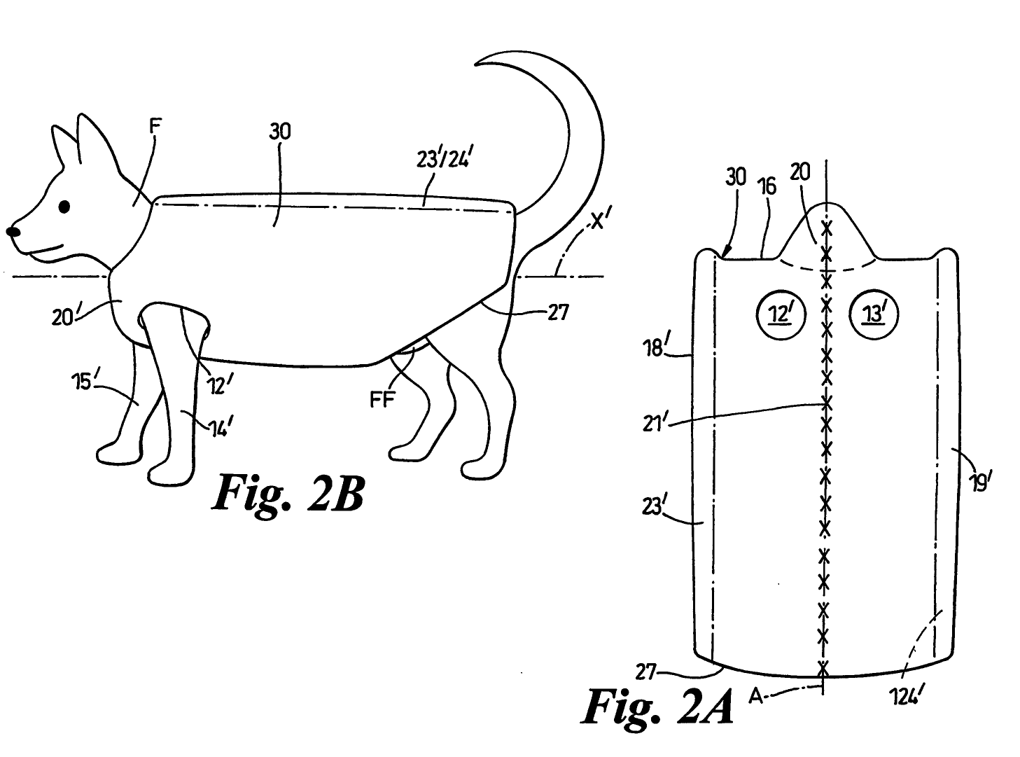

### Second Embodiment (FIGS. 2A, 2B)

These figures variously show a dressing 30 for a female dog F. The dressing 30 is broadly similar in form and in function to dressing 10 of FIGS. 1A, B. Consequently to avoid undue repetition where items in FIGS. 2A and B are similar in form and function to those of FIGS. 1A and B then the same reference letter/numerals is used in FIG. 2 with the addition of.

In this case the rear transverse side 27 of the dressing 30 is of less pronounced curvature than that of rear transverse side 17 of dressing 10. This provides for greater coverage of rear lower section FF of the female dog F than was available from dressing 20 for male dog D. This is to provide that dressing is readily applied and retained for a female dog F after a spaying operation.

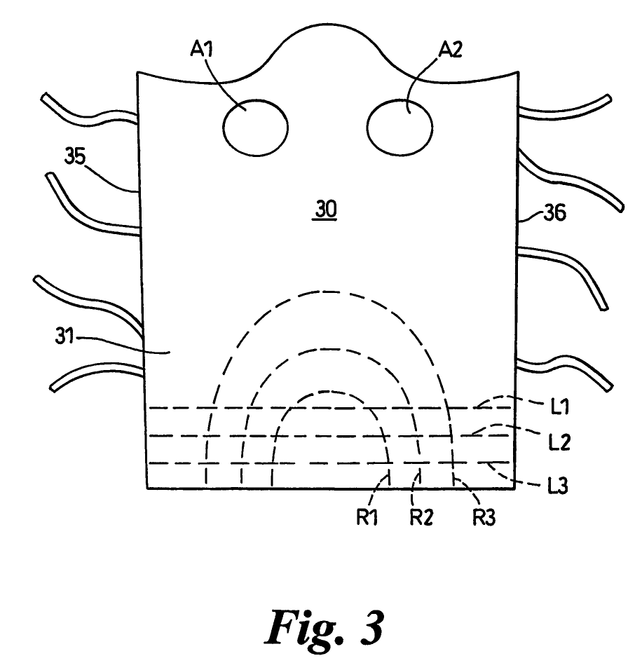

### Third Embodiment (FIG. 3)

This shows a dressing 30 having a sheet member 31 which can be readily adapted to suit a particular animal. Apertures A1, A2 are comparable in form and function to apertures 12, 13 of the first embodiment of FIG. 1A. The dressing has a front transverse side 32 similar in form and function to front side 16 of FIG. 1A. First and second lateral sides, respectively sides 35, 36 corresponding in form and function to first and second lateral sides 18, 19 of FIG. 1A. Rear portion R of the sheet member 31 is printed with broken lines, typically lines L1, L2, L3 which enable the sheet 31 to be cut to the desired length for the case of a particular animal and lines R1, R2, R3 which enable the sheet 31 to be cut out to reveal to the required extent an area of the body of the subject animal. The sheet member 31 is of a material which is readily cut without fraying. Any use of conventional stitching for whatever purpose, at least in the rear portion R, is avoided so that any cutting to length or shape does not involve cutting stitching. If stitching is to be used then it should be of a type which can be cut without the seam involved becoming unstable and unravelling. Alternatives to stitching would include gluing, tacking, moulding or stapling.

In this case the dressing 30 is provided with tape ties T to secure the dressing 30 about the body of the subject animal.

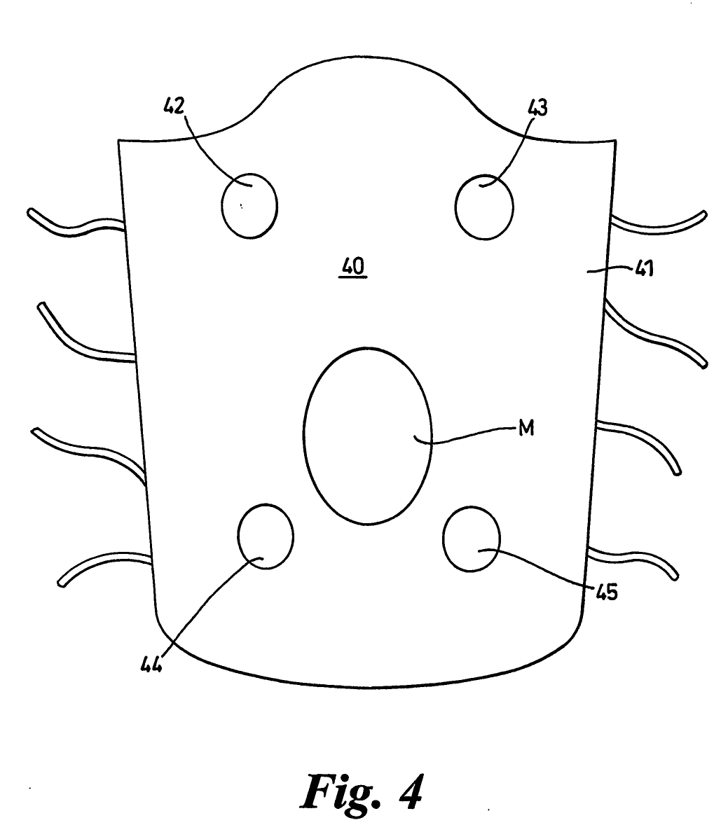

### Fourth Embodiment (FIG. 4)

This shows a dressing 40 having a sheet member 41 which in addition to apertures 42, 43 comparable in form and function to apertures 12, 13 in FIG. 1A (First Embodiment) is further provided with second apertures 44, 45 so that the dressing can be aligned by locating it over the four legs of a subject animal rather than just the front or rear pairs of legs. This embodiments shows the use of a further, central, aperture M providing access to the surface of the body typically to free feeding nipples on a female animal. In an alternative this version can be provided for in a similar manner to that shown in FIG. 3. That is to say the locations of a range of possible

The locating means of the invention can provided in a number of ways so as to locate the sheet on an animal in the vicinity of the second lateral end. Typically the locating means can be ties for locating about the tail of the animal.

The material used for the sheet members of each embodiment can be selected to achieve one or more desired ends. Typically the material will usually need to be one which is porous so as to allow the passage of air to the body beneath the sheet. However there may be cases where an impervious sheet material is required and this is readily formed into a dressing of the types described. In addition the material can be selected depending on the extent to which the dressing is intended for re-use. For re-use the material of the whole dressing should be selected so as to be readily sterilisable, such as by autoclaving or chemical means, without deterioration in the strength or functioning of the dressing.

The inner side of the sheet member can in any given case be provided with means, such as a pocket, for positively retaining further local dressing material and/or medication.

In an alternative version of the present invention at least one of the apertures or of the second apertures can be provided with a sleeve extending away from the sheet member to provide for a region of the leg of a subject animal to be sleeved.

The provision of two leg holes at the front end of the sheet provides for location based on a relatively short base length namely that corresponding to the separation between the front legs. In an alternative embodiment the sheet is as already proposed provided with two holes but in this embodiment separated on a longer base length corresponding to the separation between a front and a back leg. This longer base length can serve to provide for improved location.

### INDUSTRIAL APPLICABILITY

The dressing of the present invention can be readily manufactured in a range of sizes to cover a range of likely animal subjects. The dressing can have information on it such as instructions for use, identity of the supplier (such as a vet), advertising, address of subject. The dressings are readily packaged whether as single items or as a plurality for use in a treatment centre such as a vets or a treatment vehicle.

In a basic version of the invention it is envisaged that a sheet member similar to that shown as the rear section of FIG. 3 can be provided and a front section which, instead of being already pierced by apertures, can merely have the apertures marked for cutting. This provides for extensive use in an animal hospital or a vets of a dressing marked out after the manner of a dress pattern which is intended to accommodate a range of possible subject sizes. For a given case such a dressing is readily prepared for use for a particular size of subject animal. Typically the rear section of the ‘dress pattern’ version can accommodate marking for the selection and cutting of a second, rear, pair of apertures as described in connection with FIG. 4. The sheet member of this basic version can be provided with the complementary linkage means along the lateral sides.

1-13. (canceled)

14. A dressing for a four legged animal comprising:
a sheet member including two apertures for location at or near a front end of an animal to which a dressing is to be applied, each aperture being adapted to receive a front leg of an animal, and the sheet member (11) being bounded by:
a first transverse (front) side (16), from which the two apertures (12, 13) are spaced by an amount considerably less than an overall length of the sheet member (11);
a second transverse (rear) side (17) transverse an axis of alignment (X) and spaced on the axis (X) remotely from the first transverse side (16);
first and second lateral sides (18, 19) extending from the first transverse side (16) along the first and second sides (18, 19) of the axis of alignment (X), each the first and second sides (18, 19) extending from the first transverse side (16) to the second transverse side (17); the first lateral side (18) being disposed on one side of the axis of alignment (X) and the second later side (19) being disposed on another side of the axis of alignment (X);
locating means (20) defined by, or by way of, the sheet member (11) comprising or including a cup or recess adapted to receive a body region of an animal with its front legs projected through the two apertures (12, 13) to provide for location of the sheet member (11) on the animal so as to milit movement of the dressing relative to the animal in a direction coaxial with, or parallel to, the axis of alignment (X); and
a complementary linkage means (T) provided on the first and second lateral sides (18, 19) to enable the first lateral side (18) to be demountably coupled to the second lateral side (19) in a working configuration of the dressing (30) with the leg of the animal extending through each of the apertures (12, 13) and the first lateral side (35) to be freed from coupling with the second lateral side (36) in a standby configuration of the dressing.

15. The dressing according to claim 14, wherein second lateral side (17) is shaped so that with the dressing in the working configuration the second lateral side bounds and leaves exposed a predetermined region (DD) of a body of the animal when the dressing is in the working configuration.

16. The dressing according to claim 14, wherein the sheet (30), in a region of the second lateral side (17′), serves to define a number of possible paths (R1, R2, R3; L1, L2, L3) such as by one of marks, lines and perforations in the sheet, the paths providing for the sheet (30) to be cut to establish one of the paths as a fresh second lateral side for the dressing.

17. The dressing according to claim 14, wherein at least one of the apertures (12, 13; A1, A2) includes a sleeve extending from the aperture so that in the working configuration of the dressing, the leg of the animal extending through the aperture (12, 13, A1, A2) is surrounded, and is resiliently held, by the sleeve.

18. The dressing according to claim 14, wherein the complementary linkage means is chosen from a group consisting of at least one pair of tapes or strings which can be knotted together, a touch fastener material such as VELCRO (RTM), an adhesive and a plaster.

19. The dressing according to claim 14, wherein the dressing includes means for one of positively locating or retaining a local dressing on the sheet member (20) or for retaining medication, at a predetermined location on the sheet member (20) in a working configuration of the dressing.

20. The dressing according to claim 14, wherein the dressing (10, 30) is of a material or materials of a type able to withstand at least one autoclaving or other sterilization process to enable the dressing to be reused.

21. The dressing according to claim 14, wherein the sheet material is of a type intended from one-off disposable usage.
